(19) United States
(12) Patent Application Publication
(10) Pub. No.: US 2014/0031721 A1
(43) Pub. Date: Jan. 30, 2014

(54) Device and Method for Intra-Operative Radiation Therapy Tumor Cavity Sizing

(71) Applicant: University of Iowa Research Foundation, Iowa City, IA (US)

(72) Inventor: Timothy Waldron, Iowa City, IA (US)

(21) Appl. No.: 13/950,009

(22) Filed: Jul. 24, 2013

Related U.S. Application Data
(60) Provisional application No. 61/675,038, filed on Jul. 24, 2012.

(51) Int. Cl. A61B 5/107

(52) U.S. Cl. CPC A61B 5/1076
USPC 600/587

(57) ABSTRACT

The embodiments disclosed herein relate to various medical device components, systems and methods of use. Certain embodiments include various modular medical devices for in vivo medical procedures. More specifically, the disclosure relates to a system and methods providing re-sterilizable sizers for use in intra-operative radiation therapy.

### CROSS REFERENCE TO RELATED APPLICATIONS

This application claims priority from U.S. Provisional Application 61/675,038, filed Jul. 24, 2012, and entitled “Device and Method for Intra-Operative Radiation Therapy Tumor Cavity Sizing,” which is hereby incorporated herein by reference in its entirety.

### TECHNICAL FIELD

The embodiments disclosed herein relate to a sizing device and method for radiation cancer treatment by a mobile miniature capsule or cassette containing a radioactive source deployed internally to a patient to allow radiation emission to more precisely destroy tumors and to obtain a quality margin while not destroying unnecessary healthy tissue. Certain embodiments relate more particularly relates to various apparatuses and methods for reducing trial and error sizing by costly radiation applicators, which have limited sterilization lives.

### BACKGROUND

In treating cancers of the breast and other areas of the body, the tumor is generally removed from the tissue and biopsied, and the patient is subsequently treated with radiation for a period of days, weeks or months. Typically in breast cancer patients receiving lumpectomies, this course of radiation therapy is performed daily for approximately five weeks. This method of treating cancer applies radiation more broadly to an area of the body repeatedly, and can therefore cause a host of side effects and other undesirable outcomes.

Recent advances have sought to perform an initial, acute radiation treatment immediately following the lumpectomy, while the patient is still under anesthesia, prior to suturing. This intra-operative radiation therapy (IORT) technique thus involves the focal treatment of tissue exposed by surgery with a single dose of radiation, which overcomes the need for repeated post-operative treatments and significantly reduces any intermediate time for any remnants of the tumor to begin to re-grow.

Typically, IORT therapy is performed by way of a spherical appliance of specific size, attached to a therapeutic x-ray machine, which is placed into the surgical cavity for radiation treatment of surrounding tissue. These appliances come in a variety of sizes, and are typically extremely expensive, and generally have a lifetime of approximately 100 sterilizations.

Despite this, physicians often utilize the trial-and-error sizing method to determine the cavity size, which often results in the unnecessary re-sterilization of the unused appliances, thus shortening their overall treatment life. It is therefore of paramount importance that the physician have bespoke sizing devices and methods to eliminate the unnecessary use of the radiation appliances in trial-and-error, iterative sizing. There is thus a need in the art for improved surgical methods, systems, and devices relating to the appropriate sizing of these cavities by objects with an infinite or virtually infinite sterilization lifetime, capable of being autoclaved or otherwise sterilized as many times as is necessary without replacement. One of the principle objects of the present invention is to provide such a device.

### BRIEF DESCRIPTION OF THE DRAWINGS

FIG. 1 is a diagram showing a cutaway view of a certain exemplary embodiment of the system.

FIG. 2 is a perspective view of several sizes of an exemplary embodiment of the system.

While the invention is amenable to various modifications and alternative forms, specific embodiments have been shown by way of example in the drawings and are described in detail below. The intention, however, is not to limit the invention to the particular embodiments described. On the contrary, the invention is intended to cover all modifications, equivalents, and alternatives falling within the scope of the invention as defined by the appended claims.

### DETAILED DESCRIPTION

The various systems and devices disclosed herein relate to devices for use in medical procedures. More specifically, various embodiments relate to devices, systems and methods for the sizing of surgical cavities. It is understood that the various embodiments of the system and related methods disclosed herein can be incorporated into or used with any other known medical devices, systems and methods.

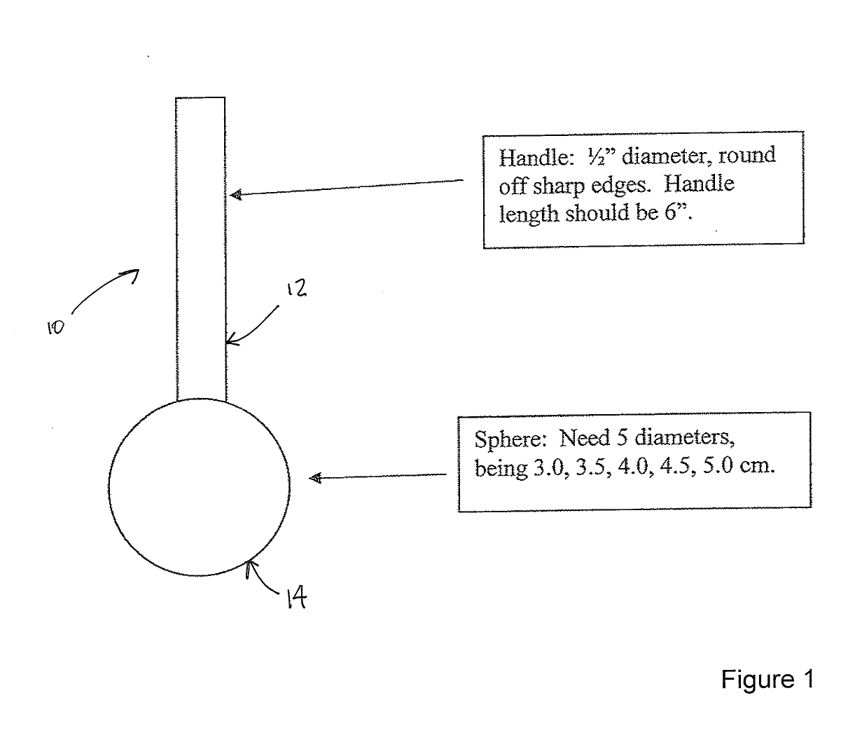

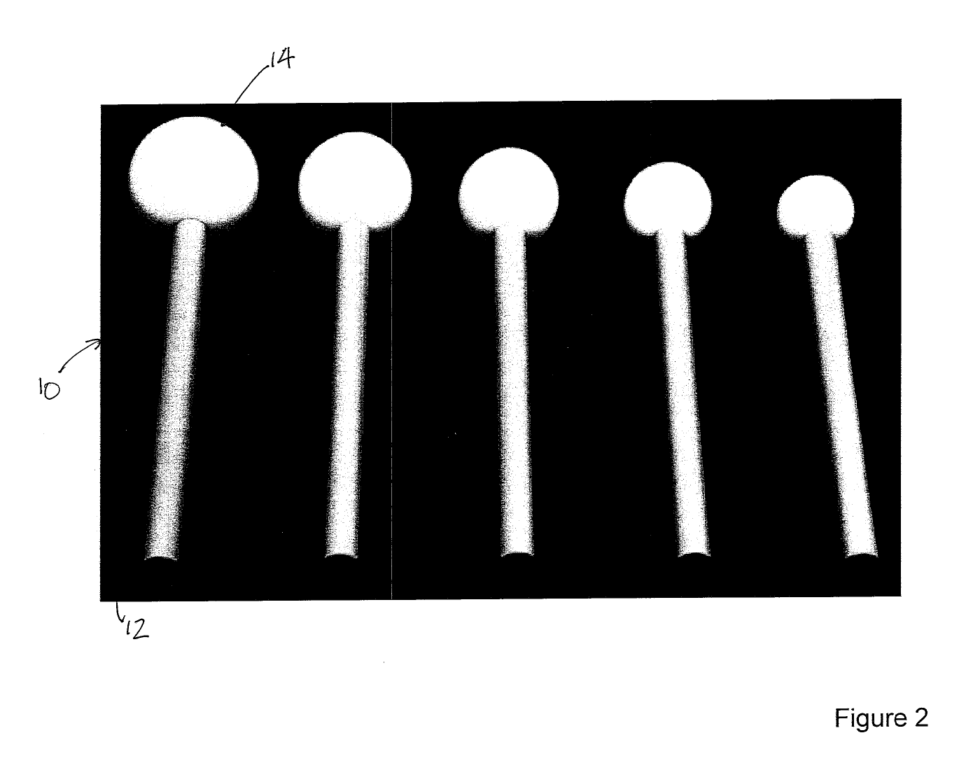

FIGS. 1-2 show various embodiments of the sizing apparatus and method. One of skill in the art would recognize that other embodiments are possible.

Turning now to the figures in detail, FIG. 1 is a schematic cutaway view of one embodiment of the sizing apparatus 10. This embodiment encompasses generally a shaft 12 affixed to a head portion 14. Although the depicted embodiment features a generally round or spherical head portion, other shapes could be used and adapted for use with specific intra-operative radiation therapy (“IORT”) units which are standard in the field.

Similarly, the shaft 12 is shown as generally quadrilateral or rectangular in the cross section. Trapezoidal and other shapes can also be utilized as necessary. In certain embodiments, the shafts are rounded so as to contour the hand of the practitioner, as would be apparent to one of skill in the art. In certain exemplary embodiments, the shaft portion and head portion are machined, cast, or otherwise formed from a single piece of material or materials, so as to have a substantially seamless joint between the head portion and shaft portion and reduce any rough edges or areas for the collection of bodily tissue, cells, bacteria or other contaminants.

As shown generally in FIG. 2, in certain exemplary embodiments of the invention, a plurality of sizing apparatuses 10 are provided, each having a head portion 14. In the depicted embodiment, five sizing apparatuses, or “sizers” are depicted. In certain exemplary embodiments, other numbers of sizers can be used, as would be readily apparent to one of skill in the art.

In exemplary embodiments, the head portions 14, are of various sizes, so as to accurately measure the diameter of the intra-operative post-lumpectomy cavity. By way of example, diameters of 1.0, 1.5, 2.0, 2.5 3.0, 3.5, 4.0, 4.5, 5.0 and 5.5 cm can be used, though other diameters and stepwise increments are also possible. Typically, resected volumes exceed 2.0 cm and are below 5 cm, though other situations may present themselves wherein sizers outside that range are needed.

In certain embodiments, the head portions 14 are fabricated from high density polyethylene and the shafts 12 are composed of anodized aluminum. In certain other embodiments, a single piece of steam-tolerant plastic may be used. Stainless steel is also possible in certain embodiments. These materials are beneficial as they are relatively low-cost, easily produced, and are capable of repeated sterilization, both under the at-hand Steris sterilization process. As would be apparent to one of skill in the art, many other materials are possible.

In use, under certain exemplary embodiments, the practitioner can select a sizer of an approximate match to the size of the cavity. The practitioner then inserts the sizer into the excision cavity of the patient. In practice—following surgery to remove a tumor or otherwise creating a cavity—the practitioner first estimates the appropriate size device 10, and inserts it into the cavity. Should it be too large to fit, the practitioner can then select a smaller size for insertion Likewise, if the fit is not snug, the practitioner can attempt to go up in size as required until the fit is appropriate.

By utilizing the system 10, the practitioner is thus able to ascertain the appropriate size for the treatment device without the use of iterative trial with the treatment device, thereby reducing the number of sterilizations required of the treatment device and reducing overall cost, as the present invention can be re-sterilized after use indefinitely, while current radioactive and other treatment devices are typically limited to approximately 100 sterilizations.

Any or all elements of the device and/or other devices or apparatuses described herein can be made from, for example, a single or multiple stainless steel alloy, nickel titanium alloys (e.g., Nitinol), cobalt-chrome alloys (e.g., ELGILOY® from Elgin Specialty Metals, Elgin, Ill.; CONICHROME® from Carpenter Metals Corp., Wyomissing, Pa.), nickel cobalt alloys (e.g., MP35N® from Magellan Industrial Trading Company, Inc., Westport, Conn.), molybdenum alloys (e.g., molybdenum TZM alloy, for example as disclosed in International Pub. No. WO 03/082363 A2, published 9 Oct. 2003, which is herein incorporated by reference in its entirety), tungsten-rhenium alloys, for example, as disclosed in International Pub. No. WO 03/082363, polymers such as polyethylene teraphathalate (PET), polyester (e.g., DACRON® from E. I. Du Pont de Nemours and Company, Wilmington, DE), poly ester amide (PEA), polypropylene, aromatic polyesters, such as liquid crystal polymers (e.g., Vectran, from Kuraray Co., Ltd., Tokyo, Japan), ultra high molecular weight polyethylene (i.e., extended chain, high-modulus or high performance polyethylene), polytetrafluoroethylene (PTFE), expanded PTFE (ePTFE), polyether ketone (PEK), polyether ether ketone (PEEK), poly ether ketone ketone (PEKK) (also poly aryl ether ketone ketone), nylon, polyether-block co-polyamide polymers (e.g., PEBAX® from ATOFINA, Paris, France), aliphatic polyether polyurethanes (e.g., TECOFLEX® from Thermedics Polymer Products, Wilmington, Mass.), polyvinyl chloride (PVC), polyurethane, thermoplastic, fluorinated ethylene propylene (FEP), absorbable or resorbable polymers such as polyglycolic acid (PGA), poly-L-glycolic acid (PLGA), polylactic acid (PLA), poly-L-lactic acid (PLLA), polycaprolactone (PCL), polyethyl acrylate (PEA), polydioxanone (PDS), and pseudo-polyamino tyrosine-based acids, extruded collagen, silicone, zinc, echogenic, radioactive, radiopaque materials, a biomaterial (e.g., cadaver tissue, collagen, allograft, autograft, xenograft, bone cement, morselized bone, osteogenic powder, beads of bone) any of the other materials listed herein or combinations thereof. Examples of radiopaque materials are barium sulfate, zinc oxide, titanium, stainless steel, nickel-titanium alloys, tantalum and gold.

Various modifications and additions can be made to the exemplary embodiments discussed without departing from the scope of the present invention. For example, while the embodiments described above refer to particular features, the scope of this invention also includes embodiments having different combinations of features and embodiments that do not include all of the above described features.

I claim:

1. A system for determining the size of excision cavities, comprising a plurality of re-sterilizable sizers, said sizers further comprising:
a. a plurality of individual shaft portions; and
b. a plurality of individual head portions of various sizes, said individual head portions affixed to the individual shaft portions for insertion into the excision cavities for assessment of the cavity size.

2. The system of claim 1, wherein the head portions are generally spherical.

3. The system of claim 1, wherein the head portion diameters are greater than 1.5 cm and less than 6.0 cm.

4. The system of claim 1, wherein the plurality of shaft portions are comprised of at least one material selected from the group consisting of anondized aluminum, high-density polyethylene, a stainless steel alloy, nickel titanium alloy, cobalt-chrome alloy, nickel cobalt alloy, molybdenum alloy, tungsten-rhenium alloy, a polymer, polyethylene teraphathalate (PET), polyester, poly ester amide (PEA), polypropylene, aromatic polyester, a liquid crystal polymer, ultra high molecular weight polyethylene, polytetrafluoroethylene (PTFE), expanded PTFE (ePTFE), polyether ketone (PEK), polyether ether ketone (PEEK), poly ether ketone ketone (PEKK), poly aryl ether ketone ketone), nylon, polyether-block co-polyamide polymer, aliphatic polyether polyurethane, polyvinyl chloride (PVC), polyurethane, thermoplastic, fluorinated ethylene propylene (FEP), absorbable or resorbable polymers such as polyglycolic acid (PGA), poly-L-glycolic acid (PLGA), polylactic acid (PLA), poly-L-lactic acid (PLLA), polycaprolactone (PCL), polyethyl acrylate (PEA), polydioxanone (PDS), and pseudo-polyamino tyrosine-based acids, extruded collagen, silicone, zinc, echogenic, radioactive, radiopaque materials, barium sulfate, zinc oxide, titanium, stainless steel, nickel-titanium alloys, tantalum and gold.

5. The system of claim 1, wherein the plurality of head portions are comprised of at least one material selected from the group consisting of anondized aluminum, high-density polyethylene, a stainless steel alloy, nickel titanium alloy, cobalt-chrome alloy, nickel cobalt alloy, molybdenum alloy, tungsten-rhenium alloy, a polymer, polyethylene teraphathalate (PET), polyester, poly ester amide (PEA), polypropylene, aromatic polyester, a liquid crystal polymer, ultra high molecular weight polyethylene, polytetrafluoroethylene (PTFE), expanded PTFE (ePTFE), polyether ketone (PEK), polyether ether ketone (PEEK), poly ether ketone ketone (PEKK), poly aryl ether ketone ketone), nylon, polyether-block co-polyamide polymer, aliphatic polyether polyurethane, polyvinyl chloride (PVC), polyurethane, thermoplastic, fluorinated ethylene propylene (FEP), absorbable or resorbable polymers such as polyglycolic acid (PGA), poly-L-glycolic acid (PLGA), polylactic acid (PLA), poly-L-lactic acid (PLLA), polycaprolactone (PCL), polyethyl acrylate (PEA), polydioxanone (PDS), and pseudo-polyamino tyrosine-based acids, extruded collagen, silicone, zinc, echogenic, radioactive, radiopaque materials, barium sulfate, zinc oxide, titanium, stainless steel, nickel-titanium alloys, tantalum and gold.

6. A method for determining the size of an excision cavity in a patient, comprising:
a. providing a plurality of re-sterilizable sizing devices; further comprising head portions and shaft portions, said shaft portions affixed to the head portions, the head portions being of a range of sizes; and
b. inserting the head portion of one or more of the sizing devices into the excision cavity of the patient to determine the approximate size of the cavity.

7. The method of claim 5, wherein the patient has undergone a lumpectomy.

8. The method of claim 5, wherein the head portions are substantially spherical.

9. The method of claim 5, wherein the head portion diameters are at least 1.5 cm and no more than 6.0 cm.

10. The method of claim 5, wherein the shaft portions are comprised of at least one material selected from the group consisting of anondized aluminum, high-density polyethylene, a stainless steel alloy, nickel titanium alloy, cobalt-chrome alloy, nickel cobalt alloy, molybdenum alloy, tungsten-rhenium alloy, a polymer, polyethylene teraphathalate (PET), polyester, poly ester amide (PEA), polypropylene, aromatic polyester, a liquid crystal polymer, ultra high molecular weight polyethylene, polytetrafluoroethylene (PTFE), expanded PTFE (ePTFE), polyether ketone (PEK), polyether ether ketone (PEEK), poly ether ketone ketone (PEKK), poly aryl ether ketone ketone), nylon, polyether-block co-polyamide polymer, aliphatic polyether polyurethane, polyvinyl chloride (PVC), polyurethane, thermoplastic, fluorinated ethylene propylene (FEP), absorbable or resorbable polymers such as polyglycolic acid (PGA), poly-L-glycolic acid (PLGA), polylactic acid (PLA), poly-L-lactic acid (PLLA), polycaprolactone (PCL), polyethyl acrylate (PEA), polydioxanone (PDS), and pseudo-polyamino tyrosine-based acids, extruded collagen, silicone, zinc, echogenic, radioactive, radiopaque materials, barium sulfate, zinc oxide, titanium, stainless steel, nickel-titanium alloys, tantalum and gold.

11. The method of claim 5, wherein the head portions are comprised of at least one material selected from the group consisting of anondized aluminum, high-density polyethylene, a stainless steel alloy, nickel titanium alloy, cobalt-chrome alloy, nickel cobalt alloy, molybdenum alloy, tungsten-rhenium alloy, a polymer, polyethylene teraphathalate (PET), polyester, poly ester amide (PEA), polypropylene, aromatic polyester, a liquid crystal polymer, ultra high molecular weight polyethylene, polytetrafluoroethylene (PTFE), expanded PTFE (ePTFE), polyether ketone (PEK), polyether ether ketone (PEEK), poly ether ketone ketone (PEKK), poly aryl ether ketone ketone), nylon, polyether-block co-polyamide polymer, aliphatic polyether polyurethane, polyvinyl chloride (PVC), polyurethane, thermoplastic, fluorinated ethylene propylene (FEP), absorbable or resorbable polymers such as polyglycolic acid (PGA), poly-L-glycolic acid (PLGA), polylactic acid (PLA), poly-L-lactic acid (PLLA), polycaprolactone (PCL), polyethyl acrylate (PEA), polydioxanone (PDS), and pseudo-polyamino tyrosine-based acids, extruded collagen, silicone, zinc, echogenic, radioactive, radiopaque materials, barium sulfate, zinc oxide, titanium, stainless steel, nickel-titanium alloys, tantalum and gold.

12. A method of determining the size of a cavity in a patient undergoing a surgical procedure, comprising:
a. providing at least one sizing device, said sizing device further comprising:
i. a head portion; and
ii. a shaft portion, wherein the shaft portion is affixed to the head portion;
b. establishing the size of the cavity by inserting the head portion of at least one sizing device into the cavity.

13. The method of claim 12, wherein the patient has undergone a lumpectomy.

14. The method of claim 12, wherein the head portions are substantially spherical.

15. The method of claim 12, wherein the head portion diameters are at least 1.12 cm and no more than 6.0 cm.

16. The method of claim 12, wherein the shaft portions are comprised of at least one material selected from the group consisting of anondized aluminum, high-density polyethylene, a stainless steel alloy, nickel titanium alloy, cobalt-chrome alloy, nickel cobalt alloy, molybdenum alloy, tungsten-rhenium alloy, a polymer, polyethylene teraphathalate (PET), polyester, poly ester amide (PEA), polypropylene, aromatic polyester, a liquid crystal polymer, ultra high molecular weight polyethylene, polytetrafluoroethylene (PTFE), expanded PTFE (ePTFE), polyether ketone (PEK), polyether ether ketone (PEEK), poly ether ketone ketone (PEKK), poly aryl ether ketone ketone), nylon, polyether-block co-polyamide polymer, aliphatic polyether polyurethane, polyvinyl chloride (PVC), polyurethane, thermoplastic, fluorinated ethylene propylene (FEP), absorbable or resorbable polymers such as polyglycolic acid (PGA), poly-L-glycolic acid (PLGA), polylactic acid (PLA), poly-L-lactic acid (PLLA), polycaprolactone (PCL), polyethyl acrylate (PEA), polydioxanone (PDS), and pseudo-polyamino tyrosine-based acids, extruded collagen, silicone, zinc, echogenic, radioactive, radiopaque materials, barium sulfate, zinc oxide, titanium, stainless steel, nickel-titanium alloys, tantalum and gold.

17. The method of claim 12, wherein the head portions are comprised of at least one material selected from the group consisting of anondized aluminum, high-density polyethylene, a stainless steel alloy, nickel titanium alloy, cobalt-chrome alloy, nickel cobalt alloy, molybdenum alloy, tungsten-rhenium alloy, a polymer, polyethylene teraphathalate (PET), polyester, poly ester amide (PEA), polypropylene, aromatic polyester, a liquid crystal polymer, ultra high molecular weight polyethylene, polytetrafluoroethylene (PTFE), expanded PTFE (ePTFE), polyether ketone (PEK), polyether ether ketone (PEEK), poly ether ketone ketone (PEKK), poly aryl ether ketone ketone), nylon, polyether-block co-polyamide polymer, aliphatic polyether polyurethane, polyvinyl chloride (PVC), polyurethane, thermoplastic, fluorinated ethylene propylene (FEP), absorbable or resorbable polymers such as polyglycolic acid (PGA), poly-L-glycolic acid (PLGA), polylactic acid (PLA), poly-L-lactic acid (PLLA), polycaprolactone (PCL), polyethyl acrylate (PEA), polydioxanone (PDS), and pseudo-polyamino tyrosine-based acids, extruded collagen, silicone, zinc, echogenic, radioactive, radiopaque materials, barium sulfate, zinc oxide, titanium, stainless steel, nickel-titanium alloys, tantalum and gold.
